(19) United States
(12) Patent Application Publication
(10) Pub. No.: US 2003/0119091 A1
(43) Pub. Date: Jun. 26, 2003

(54) Cell-free protein synthesis method and extract solution therefor

(76) Inventors: Toru Ezure, Osaka (JP); Shoken Higashide, Osaka (JP); Masaaki Ito, Osaka (JP)

Correspondence Address: WENDEROTH, LIND & PONACK, L.L.P., 2033 K STREET N. W., SUITE 800, WASHINGTON, DC, 20006-1021, US

(21) Appl. No.: 10/322,518

(22) Filed: Dec. 19, 2002

(30) Foreign Application Priority Data
Dec. 20, 2001 (JP) 387624/2001

(51) Int. Cl. C12P 21/06; C12N 9/00

(52) U.S. Cl. 435/68.1; 435/69.1; 435/183

(57) ABSTRACT

The present invention provides a cell-free protein synthesis method, which produces a protein from an exogenous template DNA via transcription and translation using an extract solution containing at least an extract derived from a Bombyx mori L. tissue and the exogenous template DNA, and an extract solution for cell-free protein synthesis.

### TECHNICAL FIELD OF THE INVENTION

The present invention relates to a novel cell-free protein synthesis method for synthesizing a protein from an exogenous template DNA via transcription and translation, and an extract solution therefor.

### BACKGROUND OF THE INVENTION

In recent years, genetic information of many organisms including human genome has been decoded. Under the circumstances, functional analysis of proteins corresponding to such genetic information and creation of genomic medicine have been attracting attention as postgenomic studies. Application and utilization of proteins corresponding to such genetic information for pharmaceutical products and the like requires easy syntheses of extensive kinds of proteins in a short time.

At present, expression systems using viable cells (hereinafter sometimes to be referred to as “cell-system”) of yeast, insect cell and the like by gene recombination technique have been widely used as the production methods of proteins. However, many proteins are difficult to express. For example, viable cells show a propensity toward elimination of exogenous proteins for their functional retention, and expression of cytotoxic proteins in viable cells prevents cell growth.

As a production method of a protein that does not use a cell-system, a cell-free protein synthesis has been known, which includes adding a substrate, enzyme and the like to a cell rupture and extract solution and the like to provide a wide choice of genetic information translation systems of organism in test tubes, and reconstructing a synthetic system capable of linking the necessary number of residues in a desired order of amino acids using an mRNA encoding the objective protein.

Such a cell-free protein synthesis is not easily limited unlike the above-mentioned cell-system protein synthesis, and proteins can be synthesized without killing the organism. In addition, because the production of protein does not accompany operations such as cultivation and the like, a protein can be synthesized in a short time as compared to cell-systems. Moreover, inasmuch as the cell-free protein synthesis also affords a large-scale production of proteins consisting of the amino acid sequence that the organism does not use, it is expected to be a promising expression method. As such cell-free protein synthesis, for example, methods using an extract solution of wheat germ and that of Escherichia coli have been known.

In a cell-free protein synthesis using an extract solution of wheat germ, however, the extraction process for the extract solution is generally extremely complicated.

As one example of the preparation method of an extract solution of wheat germ, JP-A-2000-236896 describes the following steps. Wheat seeds are added in a mill, ruptured and a crude germ fraction is obtained using a sieve. By flotation with a mixture of carbon tetrachloride and cyclohexane (carbon tetrachloride:cyclohexane=2.5:1), germinative embryo is recovered from the floating fractions and the organic solvent is removed by drying at room temperature. The impurities contained in the embryo fraction, such as seed coat and the like, are removed by adsorption using a static electricity charged body. Then, to completely remove a wheat albumen component from this sample, it is suspended in a 0.5% solution of NP40, a nonionic detergent, and repeatedly washed with an ultrasonic cleaner until the washing does not become cloudy. Ultrasonic cleaning is done once again in the presence of distilled water to purify the wheat germ.

The cell-free protein synthesis using an extract solution of wheat germ in this way requires complicated preparation of an extract solution, inconveniently demanding long hours and much labor.

A cell-free protein synthesis using an extract solution of Escherichia coli fails in glycosylation to a protein, because Escherichia coli is a procaryote, and cannot synthesize a glycoprotein. The sugar chain added to a protein by the above-mentioned glycosylation is considered to function as a function regulating factor of a protein itself or a protective and stabilizing factor of protein, in the form of a signal or ligand involved in the recognition and adhesion between substances or between cells. For the analysis of in vivo function of a protein to be glycosylated, a glycosylated protein (glycoprotein) should be obtained. Thus, there is a demand for a cell-free protein synthesis that permits glycosylation after translation into a protein.

### SUMMARY OF THE INVENTION

The present invention has been made to solve the above-mentioned problems and aims at providing a cell-free protein synthesis method, which facilitates preparation of a reaction mixture and which can synthesize glycoprotein.

As a result of the intensive studies made by the present inventors in an attempt to solve the above-mentioned problems, the present invention provides the following.

(1) A cell-free protein synthesis method, which comprises subjecting an extract solution containing at least an extract derived from a Bombyx mori L. tissue and an exogenous template DNA to transcription and translation to produce a protein from the exogenous template DNA.

(2) The cell-free protein synthesis method according to the above-mentioned (1), wherein the above-mentioned extract solution further contains a protease inhibitor.

(3) The cell-free protein synthesis method according to the above-mentioned (1) or (2), wherein a reaction mixture obtained by adding at least RNA polymerase, adenosine 5′-triphosphate, guanosine 5′-triphosphate, cytidine 5′-triphosphate, uridine 5′-triphosphate, creatine phosphate, creatine kinase, amino acid component and tRNA to the above-mentioned extract solution is used.

(4) A cell-free protein synthesis method, which comprises subjecting a liquid composition containing at least an extract derived from a Bombyx mori L. tissue and a protease inhibitor to transcription and translation to give a protein from an exogenous template DNA.

(5) The cell-free protein synthesis method according to the above-mentioned (4), wherein a reaction mixture obtained by adding at least exogenous template DNA, RNA polymerase, adenosine 5′-triphosphate, guanosine 5′-triphosphate, cytidine 5′-triphosphate, uridine 5′-triphosphate, creatine phosphate, creatine kinase, amino acid component and tRNA to the above-mentioned liquid composition is used.

(6) The cell-free protein synthesis method according to any of the above-mentioned (1)-(5), wherein the Bombyx mori L. tissue contains at least the silk gland of a Bombyx mori L. larva.

(7) The cell-free protein synthesis method according to any of the above-mentioned (1)-(5), wherein the Bombyx mori L. tissue contains at least a fat body of a Bombyx mori L. larva.

(8) The cell-free protein synthesis method according to any of the above-mentioned (1)-(5), wherein the Bombyx mori L. tissue contains at least the embryo of Bombyx mori L.

(9) The cell-free protein synthesis method according to the above-mentioned (6), wherein the Bombyx mori L. tissue contains at least the posterior silk gland of a Bombyx mori L. larva.

(10) An extract solution for cell-free protein synthesis, which comprises at least an extract derived from a Bombyx mori L. tissue and an exogenous template DNA.

(11) The extract solution according to the above-mentioned (10), which further comprises a protease inhibitor.

### BRIEF DESCRIPTION OF THE DRAWINGS

FIG. 1 is a graph showing an amount of luciferase synthesized in Example 1 relative to the reaction time, wherein the axis of ordinate shows the amount (ng/mL) of synthesized luciferase and the axis of abscissa shows the reaction time (min).

FIG. 2 is a graph showing an amount of luciferase synthesized in Example 2 relative to the reaction time, wherein the axis of ordinate shows the amount (ng/mL) of synthesized luciferase and the axis of abscissa shows the reaction time (min).

### DETAILED DESCRIPTION OF THE INVENTION

In the present specification, the “Bombyx mori L.” means Lepidoptera belonging to Bombycidae. In its life, it goes through the stages of “embryo” (from immediately after oviposition to immediately before hatching), “larva” (from immediately after hatch to immediately before completion of formation of cocoon (laraval stage 1-laraval stage 5)), “pupae” (from immediately before completion of formation of cocoon to immediately before eclosion), and “imago” (from immediately after eclosion to death), and “Bombyx mori L.” includes any stage over its lifetime.

Bombyx mori L. in the stage of larva after hatching of the egg alternately repeats the period of eating Mulberry to grow (instar)and the period of getting ready for moult without eating (moulting). In the larva of Bombyx mori L., the period of from hatching to the first moult is called laraval stage 1, and that from the 1st moult to the 2nd moult is called laraval stage 2, and the larva generally gets matured after 4 times of moult and in laraval stage 5 (Bombyx mori L. larva in the matured state is also called a “mature larva”). The mature larva of Bombyx mori L. has a transparent body, expectorates a silk thread to form a cocoon for pupation. After pupae, it ecloses into an imago.

The “silk gland” in the present specification refers to a pair of tubular exocrine glands which continue from spinneret located on the tip of labium on the head to culdesac on both sides of the body of Bombyx mori L. larva, and is roughly divided into an anterior silk gland, a middle silk gland and a posterior silk gland. The posterior silk gland secretes fibroin that constitutes the center portion of silk. The middle silk gland secretes sericin. The fibroin is accumulated in the middle silk gland and coated with sericin on the outer periphery, and forms a gel silk substance. This silk substance is discharged from spinneret through anterior silk gland and solidified to give silk.

The “fat body” in the present specification is distributed in any part of the body of Bombyx mori L. larva and is a white soft and flat band, belt or leaf tissue. Since fat body stores nutrition and energy source like human liver, the cell contains various substances related to the metabolism such as fat drop, protein, glycogen and the like.

The “embryo” in the present specification means a tissue of Bombyx mori L. in the state of egg.

In the present specification, by the “cell-free protein synthesis” is meant a protein synthesis by a cell-free transcription and translation system, which includes a transcription step for transcribing mRNA from an exogenous template DNA, and a translation step for reading the information of mRNA obtained in the transcription step to synthesize a protein. As used herein, the “protein” synthesized in the cell-free system by the synthesis method of the present invention encompasses any peptide having any molecular weight, which consists of plural amino acid residues, i.e., from low molecular weight peptides to high molecular weight peptides. The “protein” in the present specification includes glycosylated proteins.

### EMBODIMENT OF THE INVENTION

The present invention is explained in detail in the following.

An “extract derived from a Bombyx mori L. tissue” in the extract solution which is used for the cell-free protein synthesis method of the present invention may be derived from a tissue of Bombyx mori L. in any stage of its life (embryo, larva (laraval stage 1-laraval stage 5), pupae, imago). The Bombyx mori L. tissue is not limited to a single tissue in a single state (e.g., only posterior silk gland of Bombyx mori L. larvae in laraval stage 5), but may be derived from plural tissues in a single state (e.g., posterior silk gland and fat body of Bombyx mori L. larvae in laraval stage 5), or a single tissue in plural states (e.g., posterior silk gland of Bombyx mori L. larvae in each of laraval stage 3, laraval stage 4 and laraval stage 5). It is needless to say that it may be derived from plural tissues in plural states.

The above-mentioned “extract derived from a Bombyx mori L. tissue” does not need to be an extract from the entirety of the tissue of Bombyx mori L. (e.g., entire posterior silk gland).

The content of an extract derived from a Bombyx mori L. tissue in the extract solution of the present invention is free of any particular limitation, but it is preferably 1 mg/mL-200 mg/mL, more preferably 10 mg/mL-100 mg/mL, in a protein concentration. When the content of the extract is less than 1 mg/mL in a protein concentration, the concentration of the components essential for the action of the present invention becomes low and possibly prevents sufficient synthetic reaction, and when the content of the extract exceeds 200 mg/mL in a protein concentration, the extract solution itself has a high viscosity and makes operations difficult.

An extract solution containing the above-mentioned amount of an extract derived from a Bombyx mori L. tissue can be prepared utilizing the measurement of the protein concentration of the extract solution. The measurement of the protein concentration is conducted using a BCA Protein assay Kit (manufactured by PIERCE) by, for example, adding 0.1 mL of a sample to a reaction reagent (2 mL), reacting the mixture at 37° C. for 30 min and measuring the absorbance at 562 nm, as generally done in this field. As a control, bovine serum albumin (BSA) is generally used.

The above-mentioned Bombyx mori L. tissue desirably contains at least one of silk gland of Bombyx mori L. larva, fat body of Bombyx mori L. larva and embryo of Bombyx mori L. Whether or not an extract derived from at least the posterior silk gland of Bombyx mori L. larva, fat body of Bombyx mori L. larva and embryo of Bombyx mori L. is contained in an extract solution can be determined by, for example, an isozyme analysis of aldolase (Nagaoka et al., (1995), Insect Biochem Mol Biol. 25, 819-825).

It is preferable that at least an extract derived from the silk gland, particularly the posterior silk gland of Bombyx mori L. larvae be contained, because an extract solution for cell-free protein synthesis having a particularly superior advantage, that a large amount of protein can be synthesized in a short time, can be afforded.

An extract of a fat body derived from Bombyx mori L. larva is preferable because an extract solution for cell-free protein synthesis can be realized, which has a particularly superior advantage that a fat body consisting of soft tissues can be mashed in a short time, as a result of which an extract solution can be easily prepared. Whether or not a fat body is contained in an extract solution can be determined by, besides the above-mentioned isozyme analysis, detecting SP-1, SP-2 and the like, which are proteins derived from a fat body, by applying the extract solution to sodium dodecyl sulfate-polyacrylamide gel electrophoresis (SDS-PAGE).

An extract derived from embryo of Bombyx mori L. is preferable because an extract solution for cell-free protein synthesis can be realized, which has a particularly superior advantage that, because an embryo is a single individual, a step for enucleation is not necessary, unlike other tissues, as a result of which an extract solution can be prepared easily. Whether or not an embryo of Bombyx mori L. is contained in an extract solution can be determined by, besides the above-mentioned isozyme analysis, detecting 30K, ESP, Vitellin(H), Vitellin(L) and the like, which are proteins derived from an embryo, by applying the extract solution to sodium dodecyl sulfate-polyacrylamide gel electrophoresis (SDS-PAGE).

When the extract is derived from the posterior silk gland or fat body of Bombyx mori L. larva, any of laraval stage 1-laraval stage 5 of Bombyx mori L. larvae can be used for the present invention without any particular limitation. The posterior silk gland and fat body are preferably derived from Bombyx mori L. larvae in laraval stage 5. This has an advantage because the posterior silk gland and fat body of Bombyx mori L. larva in laraval stage 5 are the most mature from among those in laraval stage 1-laraval stage 5, and the use thereof enables synthesis of a large amount of protein in a short time as compared to synthesis using larvae in other laraval stages.

Particularly, the extract solution of the present invention preferably contains the posterior silk gland of Bombyx mori L. larvae in laraval stage 5, particularly an extract of the posterior silk gland of Bombyx mori L. larvae at day 3-day 7 of laraval stage 5, as an essential component, because silk fibroin, which is a main component of silk, is actively made and the Bombyx mori L. larva in this period has high protein synthesis capability.

The extract solution used for the present invention contains an exogenous template DNA as an essential component, along with the above-mentioned extract derived from a Bombyx mori L. tissue. The exogenous template DNA may be a cyclic DNA such as plasmid DNA and the like or a linear DNA such as PCR product and the like. The above-mentioned exogenous template DNA means a template DNA that is not derived from a Bombyx mori L. tissue, which contains at least a base sequence encoding the object protein and a promoter sequence located at the 5′ upstream thereof. The exogenous template DNA to be used for the present invention is not particularly limited as regards the protein (including peptide) it encodes as long as it is a template DNA not derived from a Bombyx mori L. tissue. It may have a base sequence encoding a protein that becomes cytotoxic in a viable cell, or may have a base sequence encoding a glycoprotein. The promoter sequence for the exogenous template DNA to be used in the present invention is not particularly limited and is exemplified by conventionally known T7 promoter sequence, SP6 promoter sequence, T3 promoter sequence and the like.

The exogenous template DNA to be used in the present invention is not particularly limited as regards the number of bases thereof, and the whole template DNAs do not need to contain the same number of bases as long as the objective protein can be synthesized. In addition, the exogenous template DNA may have plural bases that are deleted, substituted, inserted or added as long as it is a homologous sequence capable of synthesizing the objective protein. Whether a template DNA contained in an extract solution is an exogenous template DNA or a template DNA derived from a Bombyx mori L. tissue can be determined by extracting an extract solution with phenol-chloroform to give a template DNA therein, and analyzing a base sequence thereof.

In addition, an exogenous template DNA to be used in the present invention preferably has a terminator sequence on the 3′ downstream of a base sequence encoding the above-mentioned objective protein, which functions to terminate transcription, and/or a poly A sequence from the aspect of the stability and the like of synthesized mRNA. Examples of the above-mentioned terminator sequence include conventionally known T7 terminator sequence, SP6 terminator sequence, T3 terminator sequence and the like.

The above-mentioned extract solution preferably contains 1 μg/mL-10 mg/mL, more preferably 10 μg/mL-1000 μg/mL, of the exogenous template DNA, in view of the rate of the protein synthesis. When the amount of the exogenous template DNA is less than 1 μg/mL, the exogenous template DNA becomes unstable in the extract solution, and when it exceeds 10 mg/mL, viscosity becomes high to make operability poor. When the amount of the exogenous template DNA is less than 1 μg/mL or above 10 mg/mL, the rate of protein synthesis using this DNA tends to become lower.

By synthesizing a protein from an exogenous template DNA via transcription and translation using an extract solution containing an extract derived from a Bombyx mori L. tissue and an exogenous template DNA, any protein, even if a protein that becomes cytotoxic in viable cell, can be synthesized in a short time. In addition, because an extract derived from eucaryotic Bombyx mori L. is used, a glycoprotein can be synthesized in a cell-free system, and various kinds of proteins can be synthesized without particular limitation.

In addition, the extract solution to be used for the present invention can be strikingly easily prepared as compared to conventional preparation of an extract solution from a wheat germ, and efficient cell-free protein synthesis can be realized.

The cell-free protein synthesis method of the present invention is conducted using a DNA as it is as a template for a protein synthesis reaction and the transcription step is also conducted in a cell-free system. By this route, in the present invention, a process for preparation of mRNA to be used (e.g., process for synthesis of mRNA by introducing an exogenous template DNA into viable cells, or process for purification of the obtained mRNA after synthesis of mRNA by an in vitro transcription system and the like) is unnecessary, unlike a conventionally common method for synthesizing a protein from mRNA solely by translation in a cell-free system, and the reaction mixture can be prepared easily. As compared to DNA, mRNA is easily decomposed. A reaction mixture for protein synthesis using mRNA in a cell-free system is inferior in preservation stability, but since the DNA used in the present invention is not easily degraded and is stable, a stable reaction mixture can be advantageously prepared.

By the presence of a protease inhibitor, the preparation is facilitated and a protein (including glycoprotein) can be synthesized efficiently.

This is considered to be attributable to the fact that the activity of protease contained in an extract derived from a Bombyx mori L. tissue can be inhibited by a protease inhibitor, and an undesirable decomposition of an active protein in an extract by the protease can be prevented, as a result of which a protein synthesis capability that an extract derived from a Bombyx mori L. tissue has can be effectively elicited.

Such protease inhibitor is not particularly limited as long as it can inhibit the activity of protease, and, for example, phenylmethanesulfonyl fluoride (hereinafter sometimes to be referred to as “PMSF”), aprotinin, bestatin, leupeptin, pepstatin A, E-64 (L-trans-epoxysuccinyl-L-leucylamido (4-guanidino)butane), ethylenediaminetetraacetic acid, phosphoramidon and the like can be used. Because an extract solution derived from Bombyx mori L. tissue contains serine protease, the use of PMSF, which works as an inhibitor having high specificity to serine protease, is preferable among those mentioned above.

It is possible to use not only one kind of a protease inhibitor but also a mixture (protease inhibitor cocktail) of several kinds of protease inhibitors.

The content of the protease inhibitor in the above-mentioned extract solution is preferably 1 μM-50 mM, more preferably 0.01 mM-5 mM, because decomposition of the enzymes necessary for the action of the present invention can be preferably inhibited. This is because the decomposition activity of protease cannot be sufficiently suppressed when the protease inhibitor is less than 1 μM, and the protein synthesis reaction tends to be inhibited when the protease inhibitor exceeds 50 mM.

In the cell-free protein synthesis of the present invention, a reaction mixture obtained by adding at least RNA polymerase, adenosine 5′-triphosphate, guanosine 5′-triphosphate, cytidine 5′-triphosphate, uridine 5′-triphosphate, creatine phosphate, creatine kinase, amino acid component and tRNA to an extract solution containing at least the above-mentioned extract derived from a Bombyx mori L. tissue and an exogenous template DNA, and preferably further containing a protease inhibitor, is preferably used.

The above-mentioned reaction mixture is preferably prepared such that the above-mentioned extract solution is contained in a proportion of 10(v/v)%-80(v/v)%, particularly 30(v/v)%-60(v/v)%.

That is, an extract derived from a Bombyx mori L. tissue of 0.1 mg/mL-160 mg/mL, more preferably 3 mg/mL-60 mg/mL, in a protein concentration, is contained relative to the entirety of the above-mentioned reaction mixture. When the content of the extract is less than 0.1 mg/mL or above 160 mg/mL in a protein concentration, the reaction rate of the protein tends to become lower.

An exogenous template DNA is preferably contained in a proportion of 0.1 μg/mL-8000 μg/mL, preferably 3 μg/mL-600 μg/mL, relative to the entirety of the reaction mixture. When the content of exogenous template DNA is less than 0.1 μg/mL or above 8000 μg/mL, the synthesis rate of the protein tends to become lower.

The RNA polymerase to be used in the present invention can be appropriately selected depending on a promoter sequence that an exogenous template DNA has. For example, when an exogenous template DNA has a T7 promoter sequence, a T7 RNA polymerase recognizing the sequence is preferably used. When an exogenous template DNA has a SP6 or T3 promoter sequence, SP6 RNA polymerase or T3 RNA polymerase is preferably used, respectively.

The RNA polymerase is preferably contained in an amount of 0.01 U/.L-100 U/.L, more preferably 0.1 U/.L-10 U/.L, from the aspect of the rate of mRNA synthesis and the rate of protein synthesis in the reaction mixture. When the content of RNA polymerase is less than 0.01 U/.L, the amount of synthesized mRNA becomes small, as a result of which the rate of protein synthesis tends to fall, whereas when the content of RNA polymerase exceeds 100 U/.L, it tends to inhibit protein synthesis reaction.

The adenosine 5′-triphosphate (hereinafter sometimes to be referred to as “ATP”) is preferably contained in the reaction mixture in a proportion of 0.01 mM-10 mM, more preferably 0.1 mM-5 mM, in view of the rate of protein synthesis. When ATP is contained in a proportion of less than 0.01 mM or above 10 mM, the synthesis rate of the protein tends to become lower.

The guanosine 5′-triphosphate (hereinafter sometimes to be referred to as “GTP”) is preferably contained in the reaction mixture in a proportion of 0.01 mM-10 mM, more preferably 0.1 mM-5 mM, in view of the rate of protein synthesis. When GTP is contained in a proportion of less than 0.01 mM or above 10 mM, the synthesis rate of the protein tends to become lower.

The cytidine 5′-triphosphate (hereinafter sometimes to be referred to as “CTP”) is preferably contained in the reaction mixture in a proportion of 0.01 mM-10 mM, more preferably 0.1 mM-5 mM, in view of the rate of protein synthesis. When CTP is contained in a proportion of less than 0.01 mM or above 10 mM, the synthesis rate of the protein tends to become lower.

The uridine 5′-triphosphate (hereinafter sometimes to be referred to as “UTP”) is preferably contained in the reaction mixture in a proportion of 0.01 mM-10 mM, more preferably 0.1 mM-5 mM, in view of the rate of protein synthesis. When UTP is contained in a proportion of less than 0.01 mM or above 10 mM, the synthesis rate of the protein tends to become lower.

The creatine phosphate in the reaction mixture is a component for continuous synthesis of protein and added for regeneration of ATP and GTP. The creatine phosphate is preferably contained in the reaction mixture in a proportion of 1 mM-200 mM, more preferably 10 mM-100 mM, in view of the rate of protein synthesis. When creatine phosphate is less than 1 mM, sufficient amounts of ATP and GTP may not be regenerated easily. As a result, the rate of protein synthesis tends to become lower, and when creatine phosphate exceeds 200 mM, it acts as an inhibitory substance and the synthesis rate of the protein tends to become lower.

The creatine kinase in the reaction mixture is a component for continuous synthesis of protein and added along with creatine phosphate for regeneration of ATP and GTP. The creatine kinase is preferably contained in the reaction mixture in a proportion of 1 μg/mL-1000 μg/mL, more preferably 10 μg/mL-500 μg/mL, in view of the rate of protein synthesis. When creatine kinase is less than 1 μg/mL, regeneration of sufficient amounts of ATP and GTP becomes difficult. As a result, the rate of protein synthesis tends to become lower, and when creatine kinase exceeds 1000 μg/mL, it acts as an inhibitory substance and the synthesis rate of the protein tends to become lower.

The amino acid component in the reaction mixture contains at least 20 kinds of amino acids, i.e., valine, methionine, glutamic acid, alanine, leuicine, phenylalanine, glycine, proline, isoleucine, tryptophan, asparagine, serine, threonine, histidine, aspartic acid, tyrosine, lysine, glutamine, cystine and arginine. This amino acid includes radioisotope-labeled amino acid. Where necessary, modified amino acid may be contained. The amino acid component generally contains almost the same amount of various kinds of amino acids.

In the present invention, the above-mentioned amino acid component is preferably contained in the reaction mixture in a proportion of 1 μm-1000 μM, more preferably 10 μM-500 μM, in view of the rate of protein synthesis. When the amount of the amino acid component is less than 1 μM or above 1000 μM, the synthesis rate of the protein tends to become lower.

The tRNA in the reaction mixture contains almost the same amount of tRNAs corresponding to the above-mentioned 20 kinds of amino acids. In the present invention, tRNA is preferably contained in the reaction mixture in a proportion of 1 μg/mL-1000 μg/mL, more preferably 10 μg/mL-500 μg/mL, in view of the rate of the protein synthesis. When the amount of the tRNA is less than 1 μg/mL or exceeds 1000 μg/mL, the rate of protein synthesis tends to become lower.

The reaction mixture in the present invention preferably further contains potassium salt, magnesium salt, dithiothreitol, RNase inhibitor, spermidine and buffer.

The above-mentioned potassium salt is free of any particular limitation as long as it does not inhibit the action of the present invention, and can be used in a general form, such as potassium acetate, potassium carbonate, potassium hydrogen carbonate, potassium chloride, dipotassium hydrogen phosphate, dipotassium hydrogen citrate, potassium sulfate, potassium dihydrogen phosphate, potassium iodide, potassium phthalate and the like, with preference given to potassium acetate. Potassium salt also acts as a cofactor in the protein synthesis reaction.

The potassium salt is preferably contained in an amount of 10 mM-500 mM, more preferably 50 mM-150 mM, in the reaction mixture in the case of monovalent potassium salt, such as potassium acetate and the like, from the aspect of preservation stability. When the content of potassium salt is less than 10 mM or more than 500 mM, the components essential for protein synthesis tend to be unstable.

The above-mentioned magnesium salt is free of any particular limitation as long as it does not inhibit the action of the present invention, and can be used in a general form such as magnesium acetate, magnesium sulfate, magnesium chloride, magnesium citrate, magnesium hydrogen phosphate, magnesium iodide, magnesium lactate, magnesium nitrate, magnesium oxalate and the like, with preference given to magnesium acetate. Magnesium salt also acts as a cofactor in the protein synthesis reaction.

The magnesium salt is preferably contained in an amount of 0.1 mM-10 mM, more preferably 0.5 mM-3 mM, in the case of divalent salt, such as magnesium acetate, and the like, in the reaction mixture in the case of divalent magnesium salt, such as magnesium acetate and the like, from the aspect of preservation stability. When the content of magnesium salt is less than 0.1 mM or more than 10 mM, the components essential for protein synthesis tend to be unstable.

The above-mentioned dithiothreitol (hereinafter sometimes to be referred to as “DTT”) is added for prevention of oxidization, and is preferably contained in an amount of 0.1 mM-100 mM, more preferably 0.2 mM-20 mM, in the reaction mixture. When the content of DTT is less than 0.1 mM or more than 100 mM, the components essential for protein synthesis tend to become unstable.

The RNase inhibitor is added to this reaction mixture to prevent RNase, which is derived from Bombyx mori L. and contaminating the extract solution, from undesirably digesting mRNA and tRNA, thereby preventing synthesis of protein, during cell-free protein synthesis of the present invention. It is preferably contained in the reaction mixture in a proportion of 0.1 U/μL-100 U/μL, more preferably 1 U/μL-10 U/μL. When the amount of the RNase inhibitor is less than 0.1 U/μL, the degradation activity of RNase often cannot be suppressed sufficiently, and when the amount of the RNase inhibitor exceeds 100 U/μL, the protein synthesis reaction is tends to be inhibited.

The above-mentioned spermidine is added to promote elongation reaction during transcription. It is preferably added to the reaction mixture in a proportion of 0.01 mM-100 mM, more preferably 0.05 mM-10 mM. When the amount of spermidine is less than 0.01 mM, the synthesis rate of mRNA becomes lower and the amount of mRNA produced becomes smaller, as a result of which the rate of protein synthesis tends to become lower. When the amount of spermidine exceeds 100 mM, it tends to inhibit protein synthesis reaction.

The above-mentioned buffer imparts a buffer capacity to an extract solution, and is added for the prevention of denaturation of an extract caused by radical change in pH of an extract solution due to the addition of an acidic or basic substance and the like. Such buffer is free of any particular limitation, and, for example, HEPES-KOH, Tris-HCl, acetic acid-sodium acetate, citric acid-sodium citrate, phosphoric acid, boric acid, MES, PIPES and the like can be used.

The buffer is preferably one that maintains the pH of the extract solution at 4-10, more preferably pH 6-8. When the pH of the extract solution is less than 4 or more than 10, the components essential for the reaction of the present invention may be denatured. From this aspect, the use of HEPES-KOH (pH 7.4) is particularly preferable among the above-mentioned buffers.

The buffer is preferably contained in an amount of 1 mM-200 mM, more preferably 5 mM-50 mM, to maintain preferable buffer capacity in the extract solution. When the content of the buffer is less than 1 mM, pH may change radically due to the addition of an acidic or basic substance, which in turn may cause denaturation of the extract, and when the content of the buffer exceeds 200 mM, the salt concentration becomes too high and the components essential for protein synthesis tend to become unstable.

The reaction mixture used for the present invention more preferably contains a glycerol. When glycerol is added, the components essential for protein synthesis can be advantageously stabilized in the protein synthesis reaction. When glycerol is added, the amount is generally 5(v/v)%-20(v/v)%.

The reaction mixture to be used for the cell-free protein synthesis method of the present invention preferably contains the aforementioned extract solution containing a protease inhibitor in a proportion of 30(v/v)%-60(v/v)%, and RNA polymerase in a proportion of 0.1 U/μL-10 U/μL, ATP in a proportion of 0.1 mM-5 mM, GTP in a proportion of 0.1 mM-5 mM, CTP in a proportion of 0.1 mM-5 mM, UTP in a proportion of 0.1 mM-5 mM, creatine phosphate in a proportion of 10 mM-100 mM, creatine kinase in a proportion of 10 μg/mL-500 μg/mL, amino acid component in a proportion of 10 μM-500 μM and tRNA in a proportion of 10 μg/mL-500 μg/mL. Moreover, it is preferably realized to contain potassium acetate in a proportion of 50 mM-150 mM, magnesium acetate in a proportion of 0.5 mM-3 mM, DTT in a proportion of 0.2 mM-20 mM, RNase inhibitor in a proportion of 1 U/μL-10 U/μL, spermidine in a proportion of 0.05 mM-10 mM, HEPES-KOH (pH 7.4) in a proportion of 5 mM-50 mM, and glycerol in a proportion of 5(v/v)%-20(v/v)%.

The cell-free protein synthesis method of the present invention is performed using the above-mentioned reaction mixture containing the extract solution of the present invention in, for example, a conventionally known low temperature thermostat bath.

The reaction temperature of the transcription step is generally 10° C.-60° C., preferably 20° C.-50° C. When the reaction temperature of the transcription step is lower than 10° C., the rate of transcription tends to become lower and when the reaction temperature of the transcription step exceeds 60° C., the components essential for the reaction tend to be denatured.

The temperature of the translation step is generally 10° C.-40° C., preferably 20° C.-30° C. When the reaction temperature of the translation step is lower than 10° C., the rate of protein synthesis tends to become lower and when the reaction temperature of the translation step exceeds 40° C., the components essential for the reaction tend to be denatured.

In the present invention, the reaction is particularly preferably carried out at a temperature in the range of 20° C.-30° C., because transcription step and translation step can be sequentially conducted in this temperature range.

The reaction time is generally 1 hr-72 hr, preferably 3 hr-24 hr, for the entire steps.

The amount of the protein synthesized by the protein synthesis method in cell-free system of the present invention can be measured by enzyme activity assay, SDS-PAGE, immunoassay and the like.

The protein to be synthesized by the cell-free protein synthesis method of the present invention is not particularly limited.

The extract solution to be used for the cell-free protein synthesis method of the present invention contains at least an extract derived from a Bombyx mori L. tissue and an exogenous template DNA as mentioned above. The present invention further provides this extract solution for the cell-free protein synthesis. The extract solution of the present invention preferably contains a protease inhibitor for the aforementioned reasons. When potassium salt, magnesium salt, DTT and buffer are further contained, the components essential for the reaction of the present invention can be advantageously maintained stable.

As the potassium salt in the extract solution, various potassium salts described above as a component of the reaction mixture, preferably potassium acetate, can be preferable used. The potassium salt is preferably contained in a proportion of 10 mM-500 mM, more preferably 50 mM-200 mM, from the same aspect of the potassium salt in the aforementioned reaction mixture.

As the magnesium salt in the extract mixture, various magnesium salts described above as a component of the reaction mixture, preferably magnesium acetate, can be preferably used. The magnesium salt is preferably contained in a proportion of 0.1 mM-10 mM, more preferably 0.5 mM-5 mM, from the same aspect of the magnesium salt in the aforementioned reaction mixture.

DTT is preferably contained in the extract solution in a proportion of 0.1 mM-10 mM, more preferably 0.5 mM-5 mM, from the same aspect of DTT in the aforementioned reaction mixture.

The buffer to be contained in the extract solution is preferably similar to those used for the aforementioned reaction mixture, and the use of HEPES-KOH (pH 7.4) is preferable for the same reasons. The buffer is preferably contained in the amount of 5 mM-200 mM, more preferably 10 mM-50 mM, from the same view as in the aforementioned buffer contained in reaction mixture.

The extract solution in the present invention is prepared by adding an exogenous template DNA to an extract derived from a Bombyx mori L. tissue, which is extracted from a Bombyx mori L. tissue using a solution for extraction. Such preparation method includes at least extraction from a Bombyx mori L. tissue, and purification is preferably applied after extraction from the Bombyx mori L. tissue. Specifically, it is preferably prepared by a preparation method including at least (i) extraction from this Bombyx mori L. tissue, (ii) gel filtration of a supernatant of a liquid product obtained by extraction in (i), and (iii) collection of fractions having an absorbance at 280 nm of not less than 10 from the extract solution after gel filtration.

In the above-mentioned treatment of (i), for example, a desired tissue is removed from Bombyx mori L. according to the conventional method using a tool such as scissors, pincette, scalpel and the like. The amount of the tissue to be used for the extraction to be mentioned below, which was obtained by this removal, is free of any particular limitation, but it is generally in the range of 1 g-100 g.

Then, the removed tissue is frozen with, for example, liquid nitrogen, mashed in a mortar frozen at −80° C., and extracted with a solution for extraction. As the solution for extraction to be used here can be a conventionally known buffer solution generally used for extraction, but preferably one containing a protease inhibitor, a potassium salt, a magnesium salt, DTT and a buffer. Particularly preferably, a solution for extraction containing 0.1 mM-1 mM of PMSF, 50 mM-200 mM of potassium acetate, 0.5 mM-5 mM of magnesium acetate, 0.5 mM-5 mM of DTT and 5 mM-50 mM of HEPES-KOH (pH 7.4) is obtained.

In the treatment of (ii), the liquid product obtained by extraction in (i) is applied to centrifugal separation. The centrifugal separation is conducted under the conditions generally employed in this field (10000×g-50000×g, 0° C.-10° C., 10 min-60 min), the supernatant is recovered and again subjected to centrifugal separation under the above-mentioned conditions. The supernatant after centrifugal separation is applied to gel filtration, wherein, as the gel filtration, for example, desalting column PD-10 (manufactured by Amersham Biosciences) can be preferably used. According to a conventional method, the column is equilibrated with a buffer solution for gel filtration, a sample is fed, and the mixture is eluted with the above-mentioned buffer solution for gel filtration. The above-mentioned buffer solution for gel filtration is preferably the above-mentioned solution for extraction supplemented with glycerol. Using this, the components essential for protein synthesis are beneficially stabilized. Glycerol only need to be added at generally 5(v/v)%-40(v/v)% (preferably 20(v/v)%).

The filtrate (0.1 mL-1 mL) obtained by gel filtration is used as one fraction, as in general gel filtration, and 0.4 mL-0.6 mL is preferably used as one fraction for efficient collection of fractions having high protein synthesis capability.

In the treatment of (iii), a fraction showing an absorbance at 280 nm of not less than 10 is separated from the filtrate after gel filtration. This step includes, for example, measurement of the above-mentioned absorbance at 280 nm of each fraction using instruments such as Ultrospec 3300pro (manufactured by Amersham Biosciences) and the like and collection of fractions having the absorbance of not less than 10. An exogenous template DNA is added to the fraction(s) obtained in this way to give the extract solution. An exogenous template DNA is added such that the content of the exogenous template DNA is within a preferable range for the above-mentioned extract solution of the present invention. That is, exogenous template DNA is added such that it is preferably contained in a proportion of 1 μg/mL-10 mg/mL, more preferably 10 μg/mL-1000 μg/mL, in the extract solution. The extract solution in the present invention may naturally be one that is obtained by adding an exogenous template DNA to a mixture of plural fractions having the above-mentioned absorbance at 280 nm of not less than 10.

To obtain an extract solution containing a desired amount of the above-mentioned extract, extraction of a plural number of Bombyx mori L. bodies is generally necessary. The number of Bombyx mori L. to be subjected to the extraction varies depending on the condition and interindividual difference found in Bombyx mori L. to be used. For example, as larva approaches the time of cocoon formation, however, a smaller number of Bombyx mori L. larvae suffice for obtaining the same amount of extract due to maturation of the tissues. Because the silk gland particularly remarkably grows daily in Bombyx mori L. larva at laraval stage 5, for example, the same amount obtained from about 30 Bombyx mori L. larvae at day 1 in the laraval stage 5 can be obtained from about 6 or 7 Bombyx mori L. larvae at day 7 in the laraval stage 5.

It is preferable that the extract solution of the present invention be obtained by the above-mentioned preparation method, because the aforementioned advantages are afforded, but it does not need to be always obtained by the above-mentioned preparation method.

In addition, the present invention also provides a cell-free protein synthesis method for producing a protein from an exogenous template DNA via transcription and translation using a liquid composition containing at least an extract derived from a Bombyx mori L. tissue and a protease inhibitor. The extract derived from a Bombyx mori L. tissue and the protease inhibitor contained in this liquid composition are the same as those mentioned above with regard to the extract solution in the present invention. The liquid composition of the present invention preferably also contains a potassium salt, a magnesium salt, DTT and a buffer as mentioned above except that the exogenous template DNA is not contained. When cell-free protein synthesis reaction is performed using such liquid composition, it is performed in the same manner as preparation of the reaction mixture using an extract solution to be mentioned above, except further addition of exogenous template DNA to the reaction mixture.

### EXAMPLES

The present invention is explained in more detail in the following by referring to Examples. These are mere examples and do not limit the present invention in any way.

### Example 1

Preparation of Extract Solution Derived from Posterior Silk Gland of Bombyx mori L. Larvae

The posterior silk gland (3.07 g) was enucleated from 15 Bombyx mori L. larvae at day 4 of laraval stage 5 using scissors, pincette and scalpel, mashed in a mortar frozen at −80° C., and extracted using a solution for extraction having the following composition.

[Composition of Solution for Extraction]

20 mM HEPES-KOH (pH 7.4)

100 mM potassium acetate

2 mM magnesium acetate

2 mM DTT

0.5 mM PMSF

After extraction, the obtained liquid product was subjected to centrifugal separation in a centrifuge (himac CR20B3 (manufactured by Hitachi Koki Co., Ltd.)) under the conditions of 30000×g, 30 min, 4° C.

After centrifugal separation, only the supernatant was isolated and subjected again to centrifugal separation under the conditions of 30000×g, 10 min, 4° C. After centrifugal separation, only the supernatant was isolated. A solution for extraction containing 20% glycerol was applied to a desalting column PD-10 (manufactured by Amersham Biosciences) to equilibrate the column, the supernatant was fed and eluted with the above-mentioned solution for extraction for gel filtration.

The fraction of the filtrate after gel filtration was measured for an absorbance at 280 nm using a spectrophotometer (Ultrospec 3300pro, manufactured by Amersham Biosciences) and fractions having an absorbance of not less than 10 were collected. Thereto was added 40 μg/mL of exogenous template DNA to give an extract solution for cell-free protein synthesis derived from the posterior silk gland of Bombyx mori L. larvae at laraval stage 5. As the exogenous template DNA, one prepared according to the steps of the following (2) was used.

The obtained extract solution was measured for protein concentration using a BCA Protein assay Kit (manufactured by PIERCE). First, a sample (0.1 mL) was added to a reaction reagent (2 mL) and they were reacted at 37° C. for 30 min and absorbance at 562 nm was measured using a spectrophotometer (Ultrospec 3300pro, manufactured by Amersham Biosciences). BSA was used as a control and a calibration curve was drawn.

The content of the posterior silk gland of Bombyx mori L. larva in the extract solution was 17.5 mg/mL in a protein concentration.

(2) Preparation of Exogenous Template DNA

According to the following steps, an exogenous template DNA was prepared.

First, using luciferase T7 control DNA attached to the TNT T7 Coupled-Reticulocyte Lysate System (manufactured by Promega), Escherichia coli JM109 (manufactured by Toyo Boseki Kabushiki Kaisha) was transformed by a conventional method. Escherichia coli after transformation was cultured in LB medium (80 ml) at 37° C. for 12 hr. Plasmid DNA was prepared from the obtained cells using Plasmid Midi Kit (manufactured by QIAGEN) following the protocol.

(3) Protein Synthesis in Cell-Free System

Using extract solution prepared in the above-mentioned (1), a reaction mixture having the following composition was prepared.

[Composition of the Reaction Mixture]

50(v/v)% extract solution (exogenous template DNA in the reaction mixture: 20 μg/mL)

40 mM HEPES-KOH (pH 7.4)

100 mM potassium acetate

1 mM magnesium acetate

10 mM DTT

10(v/v)% glycerol

0.2 mM ATP

0.2 mM GTP

0.2 mM UTP

0.2 mM CTP

25 mM creatine phosphate

400 μg/mL creatine kinase

200 μM amino acid (20 kinds)

0.1 mM spermidine

1 U/μL RNase inhibitor

200 μg/mL tRNA

1 U/μL T7 RNA polymerase

ATP (manufactured by Sigma), GTP (manufactured by Sigma), CTP (manufactured by Sigma), UTP (manufactured by Sigma), amino acid (20 kinds) (manufactured by Sigma), T7 RNA polymerase (manufactured by Promega), RNase inhibitor (manufactured by TAKARA SHUZO CO., LTD.) and tRNA (manufactured by Roche Diagnostics) were respectively used.

Using the prepared reaction mixtures, and low temperature dry block bath MG-1000 (manufactured by TOKYO RIKAKIKAI Co.) as a reaction apparatus, a synthesis reaction of protein (luciferase) was performed by the cell-free system. The amount of the reaction mixture was 25 μL. The reaction temperature was 20° C. and samples were taken for each reaction time and the amount of synthesized luciferase was measured.

The synthesized luciferase was quantified using a luciferase assay kit (E-1500, manufactured by Promega). A reaction mixture (2.5 μL) was added to a luciferase assay reagent (50 μL) and luminescence by luciferase was measured using a luminometer (Turner Designs TD-20/20, manufactured by Promega).

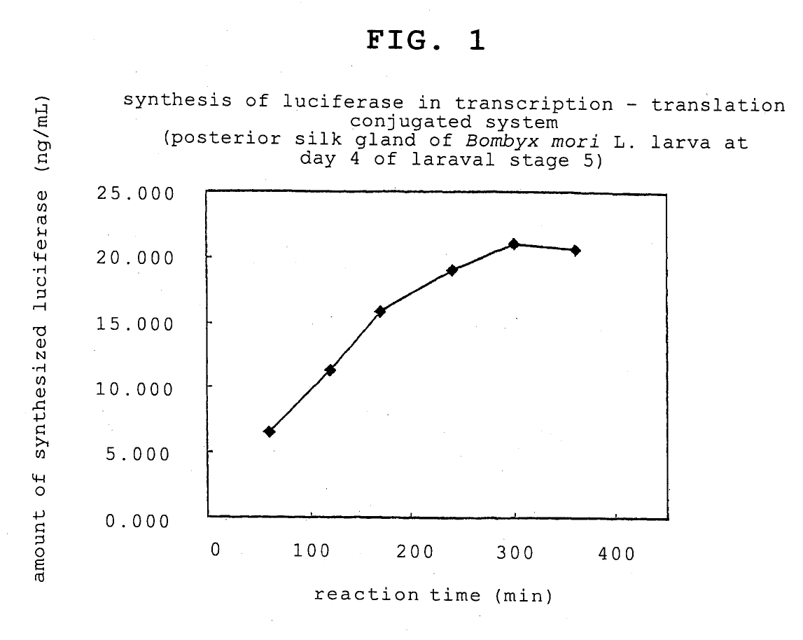

FIG. 1 is a graph showing an amount of luciferase synthesized in Example 1 relative to the reaction time. In FIG. 1, the axis of ordinate shows the amount (ng/mL) of synthesized luciferase and the axis of abscissa shows the reaction time (min).

As shown in FIG. 1, by the protein synthesis reaction in a cell-free system to produce a protein from an exogenous template DNA through transcription and translation using an extract solution containing an extract derived from the posterior silk gland of Bombyx mori L. larvae at day 4 of laraval stage 5, about 21 ng/mL of luciferase was synthesized in 300 minutes of reaction.

### Example 2

Using an extract solution prepared in the same manner as in the above-mentioned Example 1 (1) except that an exogenous template DNA (80 μg/mL) was added, a reaction mixture having the following optimized composition was prepared.

[Composition of the Reaction Mixture]

50(v/v)% extract solution (exogenous template DNA in the reaction mixture: 40 μg/mL)

10 mM HEPES-KOH (pH 7.4)

100 mM potassium acetate

0.1 mM magnesium acetate

1 mM DTT

10(v/v)% glycerol

0.2 mM ATP

0.2 mM GTP

0.2 mM UTP

0.2 mM CTP

25 mM creatine phosphate

200 μg/mL creatine kinase

40 μM amino acid (20 kinds)

0.1 mM spermidine

2 U/μL RNase inhibitor

200 μg/mL tRNA

1 U/μL T7 RNA polymerase

ATP (manufactured by Sigma), GTP (manufactured by Sigma), CTP (manufactured by Sigma), UTP (manufactured by Sigma), amino acid (20 kinds) (manufactured by Sigma), T7 RNA polymerase (manufactured by Promega), RNase inhibitor (manufactured by TAKARA SHUZO CO., LTD.) and tRNA (manufactured by Roche Diagnostics) were respectively used.

Using the prepared reaction mixtures, and low temperature dry block bath MG-1000 (manufactured by TOKYO RIKAKIKAI Co.) as a reaction apparatus, a synthesis reaction of protein (luciferase) was performed by the cell-free system. The amount of the reaction mixture was 25 μL. The reaction temperature was 20° C. and samples were taken for each reaction time and the amount of synthesized luciferase was measured.

The synthesized luciferase was quantified using a luciferase assay kit (E-1500, manufactured by Promega). A reaction mixture (2.5 μL) was added to a luciferase assay reagent (50 μL) and luminescence by luciferase was measured using a luminometer (Turner Designs TD-20/20, manufactured by Promega).

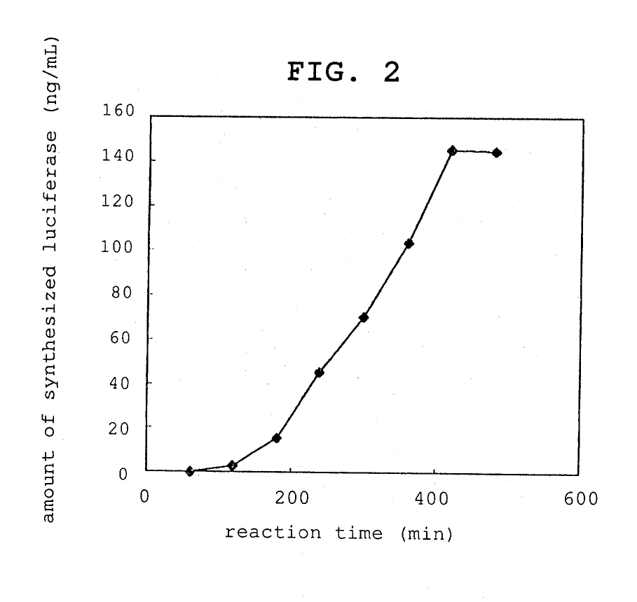

FIG. 2 is a graph showing an amount of luciferase synthesized in Example 2 relative to the reaction time. In FIG. 2, the axis of ordinate shows the amount (ng/mL) of synthesized luciferase and the axis of abscissa shows the reaction time (min).

As shown in FIG. 2, by protein synthesis reaction in a cell-free system using a reaction mixture containing an extract of the posterior silk gland derived from Bombyx mori L. larvae at day 4 of laraval stage 5, and having an optimized composition, about 146 ng/mL of luciferase was synthesized in 420 minutes of reaction.

As is clear from the foregoing explanation, the present invention provides a cell-free protein synthesis method including a transcription step, which facilitates preparation of a reaction mixture and which can synthesize a glycoprotein.

This application is based on application No. 387624/2001 filed in Japan, the contents of which are incorporated hereinto by reference.

What is claimed is:

1. A cell-free protein synthesis method, which comprises subjecting an extract solution containing at least an extract derived from a Bombyx mori L. tissue and an exogenous template DNA to transcription and translation to produce a protein from the exogenous template DNA.

2. The cell-free protein synthesis method according to claim 1, wherein the above-mentioned extract solution further contains a protease inhibitor.

3. The cell-free protein synthesis method according to claim 1, wherein a reaction mixture obtained by adding at least RNA polymerase, adenosine 5′-triphosphate, guanosine 5′-triphosphate, cytidine 5′-triphosphate, uridine 5′-triphosphate, creatine phosphate, creatine kinase, amino acid component and tRNA to the above-mentioned extract solution is used.

4. The cell-free protein synthesis method according to claim 2, wherein a reaction mixture obtained by adding at least RNA polymerase, adenosine 5′-triphosphate, guanosine 5′-triphosphate, cytidine 5′-triphosphate, uridine 5′-triphosphate, creatine phosphate, creatine kinase, amino acid component and tRNA to the above-mentioned extract solution is used.

5. A cell-free protein synthesis method, which comprises subjecting a liquid composition containing at least an extract derived from a Bombyx mori L. tissue and a protease inhibitor to transcription and translation to give a protein from an exogenous template DNA.

6. The cell-free protein synthesis method according to claim 5, wherein a reaction mixture obtained by adding at least exogenous template DNA, RNA polymerase, adenosine 5′-triphosphate, guanosine 5′-triphosphate, cytidine 5′-triphosphate, uridine 5′-triphosphate, creatine phosphate, creatine kinase, amino acid component and tRNA to the above-mentioned liquid composition is used.

7. The cell-free protein synthesis method according to claim 1, wherein the Bombyx mori L. tissue contains at least the silk gland of a Bombyx mori L. larva.

8. The cell-free protein synthesis method according to claim 5, wherein the Bombyx mori L. tissue contains at least the silk gland of a Bombyx mori L. larva.

9. The cell-free protein synthesis method according to claim 1, wherein the Bombyx mori L. tissue contains at least a fat body of a Bombyx mori L. larva.

10. The cell-free protein synthesis method according to claim 5, wherein the Bombyx mori L. tissue contains at least a fat body of a Bombyx mori L. larva.

11. The cell-free protein synthesis method according to claim 1, wherein the Bombyx mori L. tissue contains at least the embryo of Bombyx mori L.

12. The cell-free protein synthesis method according to claim 5, wherein the Bombyx mori L. tissue contains at least the embryo of Bombyx mori L.

13. The cell-free protein synthesis method according to claim 7, wherein the Bombyx mori L. tissue contains at least the posterior silk gland of a Bombyx mori L. larva.

14. The cell-free protein synthesis method according to claim 8, wherein the Bombyx mori L. tissue contains at least the posterior silk gland of a Bombyx mori L. larva.

15. An extract solution for cell-free protein synthesis, which comprises at least an extract derived from a Bombyx mori L. tissue and an exogenous template DNA.

16. The extract solution according to claim 15, which further comprises a protease inhibitor.
